SEQUENCE LISTING (12 sequences)

SEQ ID NO: 1 - DNA, 25 bp, Bacteriophage T7
aattctaatacgactcactataggg

SEQ ID NO: 2 - DNA, 20 bp, Artificial Sequence
  Basic kit overhang
gatgcaaggtcgcatatgag

SEQ ID NO: 3 - DNA, 26 bp, Artificial Sequence
  Primer for the amplification of guanylyl cyclase C RNA
ctagctggagacatcagcctggttaa

SEQ ID NO: 4 - DNA, 26 bp, Artificial Sequence
  Primer for the amplification of guanylyl cyclase C RNA
cgttggttttccaaaagttaaccaag

SEQ ID NO: 5 - DNA, 55 bp, Artificial Sequence
  Primer for the amplification of guanylyl cyclase C RNA
aattctaatacgactcactatagggagagctagctggagacatcagcctggttaa

SEQ ID NO: 6 - DNA, 55 bp, Artificial Sequence
  Primer for the amplification of guanylyl cyclase C RNA
aattctaatacgactcactatagggagagcgttggttttccaaaagttaaccaag

SEQ ID NO: 7 - DNA, 22 bp, Artificial Sequence
  Primer for the amplification of guanylyl cyclase C RNA
gtagcacctgtgaaggcctcga

SEQ ID NO: 8 - DNA, 22 bp, Artificial Sequence
  Primer for the amplification of guanylyl cyclase C RNA
cataactcaggcgactgccgga

SEQ ID NO: 9 - DNA, 26 bp, Artificial Sequence
  Detection probe for cDNA of wild type guanylyl cyclase C RNA
tggaagttttggattgtcatgtgact

SEQ ID NO: 10 - DNA, 24 bp, Artificial Sequence
  Capture probe for cDNA of guanylyl cyclase C RNA
ttgacacagaattgagctacccca

SEQ ID NO: 11 - DNA, 26 bp, Artificial Sequence
  Detection probe for cDNA of an internal control RNA
tggaatgcggtttagtgttattgact

SEQ ID NO: 12 - DNA, 20 bp, Artificial Sequence
  Detection probe for the basic kit overhang
gatgcaaggtcgcatatgag

(19) United States
(12) Patent Application Publication
(10) Pub. No.: US 2004/0018494 A1
(43) Pub. Date: Jan. 29, 2004

(54) Isothermal transcription based amplification assay for detection and quantification of guanylyl cyclase C

(76) Inventors: Roxanne N. Shurtliff, Herndon, VA (US); Eun Mi Lee, Rockville, MD (US); Joseph W. Romano, Wayne, PA (US)

Correspondence Address: BIOMERIEUX , INC., PATENT DEPARTMENT, 100 RODOLPHE STREET, DURHAM, NC, 27712, US

(21) Appl. No.: 10/202,639

(22) Filed: Jul. 25, 2002

(51) Int. Cl. C12Q 1/68; C07H 21/02

(52) U.S. Cl. 435/6; 536/23.1

(57) ABSTRACT

The present invention is directed to an isothermal transcription based assay for the detection and quantification of guanylyl cyclase C (GCC). The present invention is also directed to oligonucleotides for amplifying GCC RNA and probes for use in the detection and quantification of the amplification product. Finally, the present invention is directed to a method for detecting colon carcinoma, micrometastasis thereof and tumor recurrence by analyzing peripheral blood for the presence of GCC.

### FIELD OF THE INVENTION

The present invention relates to isothermal transcription based assays for the detection and quantification of guanylyl cyclase C (GCC). The present invention also relates to oligonucleotides for amplifying GCC RNA, as well as probes for detecting and quantitating the amplification product. The present methods are useful for detecting colon carcinoma, micrometastasis thereof and tumor recurrence by analyzing peripheral blood for the presence of GCC.

### BACKGROUND OF THE INVENTION

Colorectal cancer is the fourth most common neoplasm and the third leading cause of cancer related mortality in the United States (Waldman et al., Dis Colon Rectum 41:310-315, 1998; Jessup et al., Cancer 78:918-926, 1996). The overall mortality rate of newly diagnosed large-bowel cancer approaches 50%. Approximately 30% of patients with colorectal cancer have unresectable disease at presentation, and 33% develop metastases during the course of their disease.

Stage at diagnosis is the most important prognostic determination for patients with colorectal cancer (Cagir et al., Ann Int Med. 131:805-812, 1999; Jessup et al., supra), and it dictates the role of adjuvant chemotherapy in the disease (Cagir et al., supra; Waldman et al., supra; Fuchs and Mayer, Semin Oncol. 22:472-487, 1995). Five-year survival rates drop from 80% in patients with tumor-node-metastasis (TNM) stage II disease (who have no lymph node metastasis) to 45-50% in those with TNM stage III disease (in which lymph node metastases are present). Surgery and adjuvant chemotherapy are standard treatments for stage III disease but not for stage II disease (Leffers et al., New Engl J. Med. 339:223-228, 1998). Given the prognostic and therapeutic importance of staging, accurate histophathologic evaluation of lymph nodes to detect invasion of tumor cells has been crucial. In addition to microscopic lymph node evaluation, other techniques for staging have included intensive review of serial tissue sections, immuno-histochemical analysis to detect tumor-associated antigens, polymerase chain reaction (PCR) to detect tumor-specific mutations and reverse transcriptase (RT) PCR to detect expression of tumor-associated biomarkers (Cagir et al., supra). These techniques have not been all that successful and an easily detected biomarker expressed by colorectal tumors has been sought.

Guanylyl cyclase C (GCC) is a member of the family of receptor guanylyl cyclases, of which six members have been identified in mammals (Carrithers et al., Proc Natl Acad Sci USA. 93:14827-14832, 1996; Fulle and Garbers, Cell Biochem Funct. 12:157-165, 1994). In normal adult placental mammals, functional GCC has been identified in intestinal mucosa cells, but not in normal extraintestinal tissues (Carrithers et al., supra). Also, these receptors have been identified on human colon carcinoma cell lines in vitro (Carrithers et al., supra). Thus, GCC has been demonstrated to be expressed specifically in normal human intestinal mucosal cells and human primary and metastatic colorectal tumors. The most sensitive method for detecting expression of GCC by metastatic tumor cells in lymph nodes or other tissues is RT-PCR using GCC specific primers (Carrithers et al., supra). It has also been found that RT-PCR using GCC-specific primers is able to detect metastatic colon tumor cells undetected in routine histopathology in lymph nodes of patients being staged for colon cancer (Waldman et al., supra). In addition, RT-PCR using GCC-specific primers is able to detect recurrence of colorectal cancer in patients with stage II disease (Cagir et al., supra).

One of the advantages of an isothermal transcription based amplification method, as compared to other amplification methods such as PCR, is that by being essentially isothermal, it requires few manipulations by the experimenter. The method may be used on purified or semi-purified RNA extracts, or on cell or tissue samples with in situ amplification. In addition, if the sample contains both DNA and RNA, the use of RT/PCR requires a first step of DNAse treatment, or some method to distinguish the amplification products of mRNA- and DNA-derived PCR products is necessary. DNAse treatment prior to RT-PCR can be employed (Bitsch et al., J Infect Dis. 167:740-743, 1993; Meyer et al., Mol Cell Probes 8:261-271, 1994), but sometimes fails to remove contaminating DNA sufficiently (Bitsch et al., supra).

Thus, there is a need in the art for an isothermal amplification method to detect the presence of GCC and hence detect colon carcinoma, metastasis thereof and cancer recurrence.

### SUMMARY OF THE INVENTION

The present invention provides isothermal transcription based amplification assays for the detection and quantification of GCC. The detection assay uses primer pairs and probes for GCC. Quantitative assays use an internal control RNA (Q) that differs from the GCC RNA (also referred to herein as wild type (WT) RNA) by a small internal randomized segment. Q RNA is spiked into the sample at a known copy number at lysis and is coextracted and coamplified with GCC RNA. The resulting amplificate is then subjected to two independent hybridization reactions with probes specific for the GCC and Q amplificates. Quantitation of the GCC RNA is achieved by calculating the ratio of resulting signal of GCC to Q.

Amplification in an isothermal transcription based amplification system is achieved through the coordinated activities of three enzyme activities (reverse transcriptase, RNase H, and RNA polymerase) and two DNA oligonucleotides (referred to herein as primers) specific for the target sequence. The method starts with an RNA template and alternately synthesizes DNA and RNA. Using an RNA template, a primer, and reverse transcriptase, an RNA/DNA hybrid is generated. The RNA is degraded from the hybrid by the RNase H activity. A double stranded DNA is then generated by the reverse transcriptase using another primer, and then the double stranded DNA is used as template for large amounts of RNA synthesis by the RNA polymerase. One of the primers has, in addition to the sequences complementary to the template, additional sequences necessary for generating an RNA polymerase promoter and transcription initiation site which can be used by the RNA polymerase. The single stranded RNA product can be readily detected through the hybridization of an appropriately labeled oligonucleotide DNA probe, with or without an additional probe which can be used to immobilize the amplification product. Detection of an amplification product indicates that the target molecule (RNA) is present in the sample, and detection of specific quantities of amplification product indicate target molecules present in the sample in specific amounts.

The samples used in the methods of the present invention may be various body tissues or cells, or cells cultured in vitro from humans or other animals. In many cases, the sample is peripheral blood or cells obtained from lymph nodes. The level of CGC RNA in the sample correlates with the disease progression and is therefore useful information in the prognosis and/or management of colorectal cancer.

### DETAILED DESCRIPTION OF THE INVENTION

An isothermal transcription based assay is used for the detection and quantitation of GCC RNA. Any isothermal transcription based assay may be used with the primers and probes of the present invention. The isothermal transcription based assay of the present invention is carried out under conditions that can be readily determined by a person of ordinary skill in the art.

The preferred amplification method of the present invention is the isothermal transcription based amplification system referred to as NASBA. The NASBA method is disclosed in U.S. Pat. Nos. 5,409,818 and 5,554,527, each incorporated herein by reference. NASBA includes the use of T7 RNA polymerase to transcribe multiple copies of RNA from a template including a T7 promoter. Additional NASBA assays are also disclosed in U.S. Pat. Nos. 6,093,542 and 6,121,023, each incorporated herein by reference.

Another technique for the amplification of nucleic acid is the so-called transcription based amplification system (TAS). The TAS method is described in published PCT patent application No. WO 88/10315, incorporated herein by reference. Transcription based amplification techniques usually comprise treating target nucleic acid with two oligonucleotides one of which comprises a promoter sequence, to generate a template including a functional promoter. Multiple copies of RNA are transcribed from said template and can serve as a basis for further amplification.

Other transcription based amplification techniques are described in published European Patent application No. 408295, incorporated herein by reference. EP 408295 is primarily concerned with a two-enzyme transcription based amplification method. Transcription based amplification methods, such as the NASBA method described in published European Patent application 329822, incorporated herein by reference, are usually employed with a set of oligonucleotides, one of which is provided with a promoter sequence that is recognized by an enzyme with DNA dependent RNA polymerase activity such as, for example, T7 polymerase. Several modifications of transcription based techniques are known in the art. These modifications comprise, for example, the use of blocked oligonucleotides (that may be provided with a promoter sequence). These oligos are blocked so as to inhibit an extension reaction proceeding therefrom (U.S. Pat. No. 5,554,516, incorporated herein by reference). One or more “promoter-primers” (oligonucleotides provided with a promoter sequence) may be used in transcription based amplification techniques, optionally combined with the use of one or more oligonucleotides that are not provided with a promoter sequence.

The term “oligonucleotide” as used herein refers to a molecule comprised of two or more deoxyribonucleotides or ribonucleotides. Such oligonucleotides may be used as primers and probes.

Of course, based on the sequences of the oligonucleotides of the present invention, analogues of oligonucleotides can also be prepared. Such analogues may constitute alternative structures such as “PNA” (molecules with a peptide-like backbone instead of the phosphate sugar backbone of normal nucleic acid) or the like. It is evident that these alternative structures, representing the sequences of the present invention, are likewise part of the present invention.

The term “primer” as used herein refers to an oligonucleotide either naturally occurring (e.g., as a restriction fragment) or produced synthetically, which is capable of acting as a point of initiation of synthesis of a primer extension product which is complementary to a nucleic acid strand (template or target sequence) when placed under suitable conditions (e.g., buffer, salt, temperature and pH) in the presence of nucleotides and an agent for nucleic acid polymerization, such as DNA dependent or RNA dependent polymerase. A primer must be sufficiently long to prime the synthesis of extension products in the presence of an agent for polymerization. A typical primer contains at least 10 nucleotides in length of a sequence substantially complementary or homologous to the target sequence, but somewhat longer primers are preferred. Usually primers contain about 15-26, nucleotides, but longer primers may also be employed, especially when the primers contain additional sequences such as a promoter sequence for a particular polymerase.

Normally a set of primers will consist of at least two primers, one “upstream” (P2) and one “downstream” (P1) primer, which together define the amplificate (the sequence that will be amplified using said primers). The primers preferably have an A as the final nucleotide at the 3′-end thereof. One of the primers is understood to contain, in addition to sequences that will hybridize to the target sequence, sequences which provide promoter activity. The promoter sequences are operably attached to the 5′ end of the primer sequence. See Table 1. Most often the P1 primer will include the promoter sequence. Stretches of pyrimidines (C or T) in the first 10-12 nucleotides of the P1 primer sequence complementary to the target RNA sequence may cause abortive transcription. Thus, if no pyrimidine-rich region is available in the target RNA (i.e., C or U residues in the target hybridizing to G or A residues in the P1 primer), extra purine residues (for example, AGAG or AGAAGG) may be inserted in the P1 primer immediately after the final triplet of G residues in the T7 RNA polymerase sequence. See Table 1.

The term “promoter sequence” defines a region of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are recognized by certain bacteriophage RNA polymerases such as bacteriophage T3, T7 or SP6. Their function as a primer, e.g., the starting point for an elongation reaction, however, may be blocked, as already mentioned above, or absent in some embodiments of transcription based amplification reactions. A particularly preferred promoter sequence is the sequence of the T7 RNA polymerase promoter:

AATTCTAATACGACTCACTATAGGG (SEQ ID NO:1).

A preferred embodiment of the present invention is a combination of two oligonucleotides according to the invention, for use as a set in nucleic acid amplification.

One of the oligonucleotides may serve as an “upstream oligonucleotide”, i.e., upstream primer, while the second oligonucleotide serves as a “downstream oligonucleotide”, i.e., downstream primer, in the amplification reaction.

Preferably, the reverse transcriptase activity is provided by avian myeloblastosis virus (AMV) reverse transcriptase and the RNA polymerase is provided by T7 RNA polymerase.

One of the advantages of an isothermal transcription based amplification method, as compared to other amplification methods such as PCR, is that by being essentially isothermal, it requires few manipulations by the experimenter. However, the absence of a high temperature step does make it somewhat more difficult to find appropriate primers (see below).

The amplification method of the present invention may be applied to extracts of samples comprising nucleic acid, or whole cells or tissues for in situ amplification. The samples may be various body fluids, particularly blood, plasma, and serum, from humans. The samples may also be tissue samples from humans.

If the method is applied to extracts of samples comprising nucleic acids, the sample may be total RNA extracts (such as those described in Chomczynski and Sacchi, Anal Biochem. 162:156, 1987) or “Boom” extracts (Boom et al, J Clin Micro. 28:495-503, 1990). The method is preferably applied to “Boom extracts”.

The amplificate is detected by hybridization with an appropriately labeled oligonucleotide probe. The label may contain a radioactive moiety, a detectable enzyme, or any other moiety capable of generating a detectable signal, such as a colorimetric, fluorescent, chemiluminescent or electrochemiluminescent (ECL) signal. Blot based hybridization analysis and liquid hybridization based ECL analysis are preferably used, although other analysis systems such as ELGA (enzyme-linked gel assay) and in situ hybridization can also be used.

In one embodiment of the present invention, the amplification products are resolved by agarose gel electrophoresis, then transferred to nylon membranes and hybridized to a probe that is 5′-end labeled with ³²P using standard methods. The products are then visualized by autoradiography. In a second embodiment of the present invention, the amplification products can be detected using ELGA. In this method a probe that is specific for the amplification reaction product and conjugated at its 5′ end with horseradish peroxidase (HRP) is hybridized to the amplification product. The hybridization product is then resolved electrophoretically on a polyacrylamide gel. A calorimetric enzyme reaction allows for the visualization of the reaction product in the gel. A third embodiment of the present invention makes use of electrochemiluminescence chemistry (or ECL). This embodiment uses a biotinylated capture probe immobilized onto the surface of a streptavidin-coated magnetic bead via the biotin-avidin interaction. This system also requires an oligonucleotide detector probe, which can hybridize to an independent region of the amplification product. This detector probe is labeled with Ruthenium, the substance that is responsible for generating an ECL signal.

The quantitative method of the present invention may use one or more internal controls to monitor the efficiency of the extraction process and the amplification assay itself. The detection systems are described in detail in Romano et al. (DNA Technology 16:89-103, 1996), and van Gemen et al. (J Virol Methods 49:157-168, 1994). Methods for internal controls are described in van Gemen et al. (Reviews in Medical Virology 5:205-211, 1995).

In a preferred embodiment of the quantitative assay of the present invention, known amounts of in vitro transcribed Q RNA are spiked into the samples prior to RNA extraction, and are thereafter subjected to the same extraction and amplification procedures as the samples themselves. The Q probe is used to detect the Q amplification product and the wild type (wt) probe is used to detect the amplification product of the GCC RNA in the sample. The amount of signal from the Q amplification is then compared to the amount of signal from the wt amplification product to determine the amount of GCC RNA present in the sample.

It may also be relevant to adapt the assay for an in situ format, which would be useful in pathology studies of tissue, particularly for lymphatic tissues. If the method is to be practiced on fixed preparations for in situ analysis, the method is performed as follows. Samples may include various body fluids or tissue samples. Lymph tissue is a preferred tissue for in situ analysis. The cells are fixed and then permeabilized to optimize permeability of the cell membranes. The fixatives are those standardly used in the art for cell or tissue preparations, such as acetone and methanol, ethanol, formalin, formaldehyde, paraformaldehyde, or Permafix.RTM., and the permeabilization is done by proteinases, such as proteinase K or pepsinogen. The cells are then washed to remove all reagents that might inhibit the transcription based reaction. Permeabilization is done to the point that the cells allow entry of all necessary amplification reaction components, yet retain the targets and amplification products within the cells. In addition, cosolvents such as glycerol or DMSO may be added to optimize the NASBA reaction.

Detection of amplification products may be by direct labelling (with, for instance, biotin or digoxigenin-UTP) or by in situ hybridization with labelled probe. The direct labelling method requires that conditions can be optimized to remove unincorporated label while maintaining the amplification products.

In a particularly preferred embodiment of the present invention, the isothermal transcription based amplification method is used in concert with a particular RNA extraction technique (“Boom extraction”, Boom et al., supra), and ECL detection (electrochemiluminescence). The advantages of the system are those associated with an amplification based assay capable of providing sequence level data. Although some of these same advantages exist for the RT-PCR (i.e., increased sensitivity over ELISA, gene sequence specificity), there are advantages of NASBA for RNA over RT-PCR. These include isothermal amplification, incorporation of reverse transcription into the amplification, application to wider array of specimen types (via Boom extract), and the sensitivity and dynamic range of the ECL detection.

Boom extracts are purified preparations of DNA and RNA. The Boom method is based on the lysing and nuclease inactivating properties of the chaotropic agent guanidinium thiocyanate (GuSCN) together with the nucleic acid binding properties of silica particles or diatoms. By using size fractionated silica particles, nucleic acids, including covalently closed circular, relaxed circular, linear double-stranded DNA, single stranded DNA, tRNA, mRNA, and rRNA, can be purified from a sample in less than one hour and recovered in the original reaction vessel.

A small sample is pipetted into a reaction vessel containing a solid nucleic acid carrier and a GuSCN containing lysis buffer. Lysis of the cells occurs and the released nucleic acids bind to the carrier. The carrier-nucleic acid complexes can be separated by centrifugation. Several wash steps follow and the complexes are then dried. The nucleic acids are eluted in an aqueous low-salt buffer in the initial reaction vessel and used for the amplification reaction.

In a preferred embodiment of the present invention, amplification is achieved in a 20 μL reaction containing 5 μL of the nucleic acid extract material in 10 μL of premix (Tris (40 mM) pH 8.5; MgCl₂ (12 mM); KCl (70 mM); DTT (5 mM); dNTPs (each) (1 mM); rATP, rUTP, rCTP (2 mM); rGTP (1.5 mM); ITP (0.5 mM); DMSO (15%); P1 and P2, (0.2 μM); Sorbitol (1.5 M)). This mixture is then added to 5 μL of enzyme mix (BSA (2.1 μg/NASBA); RNase H (0.08 unit/NASBA); T7 RNA Polymerase (32 units/NASBA); and AMV-RT (6.4 units/NASBA)). (The enzyme mixture must not be vortexed). If the nucleic acid sample decreases (5 μl), then the water volume increases accordingly so that the total volume stays 15 μl when the nucleic acid is added.

The method can be carried out as follows.

1. Mix premix.

2. Add 10 μl of premix to 5 μL of nucleic acid in an EPPENDORF tube.

3. Incubate at 65° C. for 5 minutes.

4. Transfer to 41° C. heat block, incubate for 5 minutes.

5. Add 5 μl of enzyme mix.

6. Mix without vortexing.

7. Incubate at 41° C. for 5 minutes.

8. If the tops of the tubes have condensation from the cooling, they may be spun.

9. Incubate at 41° C. for 90 minutes.

10. Spin down samples and store at −20° C.

A technical challenge encountered in the development of NASBA assays is the selection of primers. It has often been the case that primers selected from sequence data, and meeting all the known requirements for primers, do not actually function in practice. In addition, in some cases primers have been developed using model systems such as in vitro transcribed RNA, virus stocks, or cell lines with very high expression of the target gene, but those primers were found to be nonfunctional when the target molecule is in a background of clinical samples. The exact mechanism underlying this problem is not understood, but is believed to arise due to the lower temperature of the NASBA reaction, which does not entirely melt secondary structure of the target molecule and/or allows nonspecific binding of primers to background nucleic acids in the sample. It is essential for the application of the NASBA system to clinical samples that the primers not be absorbed by background nucleic acids, but rather be available for specific binding to the target molecule. The problem is compounded by the inability of the low NASBA temperature to relax secondary structure in the template RNA, making proper primer annealing even more difficult. Thus, actual primers can only be developed by means of empirical investigation, and the fundamental nature of the NASBA process (i.e. low temperature) prevents accurate prediction of functional primer sets. 1

In the method of the present invention, NASBA primers were designed for GCC RNA. The primer and probe sequences were derived from the Genbank entries for this gene (Accession No. M73489). A total of four primers were designed and synthesized; there were four primer combinations (P1A and P2A (“AA”); P1A and P2B (“AB”); P1B and P2A (“BA”); and P1B and P2B (“BB”)) for the target sequence. The primers and probes are listed in Table 1.

TABLE 1

GCC Oligonucleotides

| | | |
|---|---|---|
| Oligo | Map Position* | Sequence (5′-3′) |

| | | |
|---|---|---|
| BK overhang | NA | GAT GCA AGG TCG CAT ATG AG (SEQ ID NO.: 2) |
| P1 A | 469-494 | CTA GCT GGA GAC ATC AGC CTG GTT AA (SEQ ID NO.: 3) |
| P1 B | 510-535 | CGT TGG TTT TCC AAA AGT TAA CCA AG (SEQ ID NO.: 4) |
| P1 A/Pm** | | AAT TCT AAT ACG ACT CAC TAT AGG G (AGAG)-CTA GCT GGA GAC ATC |
| | | AGC CTG GTT AA (SEQ ID NO.: 5) |
| P1B/Pm | | AAT TCT AAT ACG ACT CAC TAT AGG G (AGAG)-CGT TGG TTT TCC AAA |
| | | AGT TAA CCA AG (SEQ ID NO.: 6) |
| P2 A | 290-311 | GTA GCA CCT GTG AAG GCC TCG A (SEQ ID NO.: 7) |
| P2 B | 268-289 | CAT AAC TCA GGC GAC TGC CGG A (SEQ ID NO.: 8) |
| WT probe A | 432-457 | TGG AAG TTT TGG ATT GTC ATG TGA CT (SEQ ID NO.: 9) |
| Capture probe B | 398-421 | TTG ACA CAG AAT TGA GCT ACC CCA (SEQ ID NO.: 10) |
| Q probe*** | NA | TGG AAT GCG GTT TAG TGT TAT TGA CT (SEQ ID NO.: 11) |
| BK detection probe | NA | GAT GCA AGG TCG CAT ATG AG (SEQ ID NO.: 12) |

*Map position corresponds to base 1 as the A in the initiator codon.
**Pm indicates the T7 RNA polymerase promoter sequence (SEQ ID NO: 1), as shown in italics.
***The italicized nucleotides in the Q probe indicate the substitution with regard to the WT probe A.

The present invention is further detailed in the following Examples, which are offered by way of illustration and are not intended to limit the invention in any manner. Standard techniques well known in the art or the techniques specifically described below are utilized.

### EXAMPLE 1

NASBA—Initial Evaluation

Two P1 and two P2 primers were designed for the GCC template as listed in Table 1. RNA was extracted by the method of Chomczynski and Sacchi from a colorectal adenocarcinoma cell line, HT-29. Four possible combinations of the primers, AA, AB, BA and BB, were used in standard NASBA reactions with 5 ng of total RNA. The initial evaluation used the P2 overhang method, which employs a generic overhang on the 5′end of the P2 primers. This allows for the Basic Kit (BK) overhang to be incorporated into the amplification product, thus being the target for the BK detection probe.

Amplification was achieved in a 20 ul reaction containing 5 ul of the nucleic acid extract material in 10 ul of premix [Tris (40 mM) pH 8.5; MgCl2 (12 mM); KCl (70 mM); DTT (5 mM); dNTPs (each) (1 mM); rATP, rUTP, rCTP (2 mM); rGTP (1.5 mM); ITP (0.5 mM); DMSO (15%); P1 and P2 (0.2 uM) Sorbitol (1.5 M)]. This is then added to 5 ul of enzyme mix [BSA (2.1 ug/NASBA); RNase H (0.08 units/NASBA); T7 RNA Polymerase (32 units/NASBA); and AMV-RT (6.4 units/NASBA)].

The NASBA products were detected using ECL detection with the BK detection probe and the wild type (WT) probe A used as a capture probe. The initial analysis indicated that all four primer combinations were functional in NASBA amplification, although primer pair AA was better than others. The results are shown in Table 2.

TABLE 2

| Primer Set | 5 ng HT-29 total RNA |
|---|---|
| AA | + |
| AB | + |
| BA | + |
| BB | + |

### EXAMPLE 2

The sensitivity of the GCC NASBA assay was determined using primer sets AA and AB. HT-29 cells and COLO 205 cells, a colorectal adenocarcinoma cell line, were diluted and extracted by the Boom method (Boom et al, J Clin. Micro.: 28, No.3, March 1990, p.495-503). The extracts were amplified using primer set AA, after which the amplified product was detected by the ECL detection method using the BK detection probe and the WT probe A as a capture probe. The results in Table 3 show that the NASBA based method is sensitive for detecting GCC RNA.

TABLE 3

| Cell Number | HT-29 AA | HT-29 AB | Colo-205 AA |
|---|---|---|---|
| 9 × 10\hat{ }4 | + | + | + |
| 9 × 10\hat{ }3 | + | + | + |
| 9 × 10\hat{ }2 | + | + | − |
| 9 × 10\hat{ }1 | + | − | − |
| 9 × 10\hat{ }0 | − | − | − |

### EXAMPLE 3

The specificity of the GCC NASBA assay was determined using primer set AB. Two prostate carcinoma cell lines (LNCaP and PC-3) and normal human PBMC were used as negative controls. The cells were extracted by the Boom method, after which the extract was amplified using primer set AB. The amplified product was then detected by the ECL detection method using the BK detection probe and the WT probe A used as a capture probe. The results in Table 4 show that the NASBA assay is specific for its intended target.

TABLE 4

| Cell Number/Sample | GCC ECL Result |
|---|---|
| 9 × 10\hat{ }4 HT-29 (positive control) | + |
| 10\hat{ }5 LNCaP | − |
| 10\hat{ }5 PC-3 | − |
| 7 × 10\hat{ }4 PBMC | − |
| 7 × 10\hat{ }4 PBMC | − |
| Water | − |

### EXAMPLE 4

The P1 primers were used with the P2 primers without the BK overhang. T84 cells, a colorectal carcinoma cell line, were used as a template to determine the sensitivity of the NASBA assay using primer sets AA and AB. The cells were extracted by the Boom method, the extract amplified, and the amplified product was detected using the capture probe B and the WT probe A as a detection probe. The results in Table 5 show that the NASBA assay, when using primer sets lacking the BK overhang, can detect GCC RNA in a selected cell line at a highly sensitive level.

TABLE 5

| Cell Number | AA | AB |
|---|---|---|
| 9 × 10\hat{ }4 | + | + |
| 9 × 10\hat{ }3 | + | + |
| 9 × 10\hat{ }2 | + | + |
| 9 × 10\hat{ }1 | + | + |
| 9 × 10\hat{ }0 | + | + |
| Water | − | − |

### EXAMPLE 5

A quantitative assay for GCC RNA was developed as follows. The cDNA for GCC was cloned and used for the production of in vitro transcribed RNA and quantified for copy number using ultraviolet spectrophotometry. The RNA was then diluted and amplified using AA or AB primer sets. The results in Table 6 show that the sensitivity of the assay is between 5 and 50 copies of in vitro transcribed RNA.

TABLE 6

| Input | | | | |
|---|---|---|---|---|
| GCC | | | | |
| RNA | Primer Set and Template | | | |
| copies | AA:WT RNA | AA:Q RNA | AB: WT RNA | AB: Q RNA |
| 5 × 10\hat{ }5 | + | + | + | + |
| 5 × 10\hat{ }4 | + | + | + | + |
| 5 × 10\hat{ }3 | + | + | + | + |
| 5 × 10\hat{ }2 | + | + | + | + |
| 5 × 10\hat{ }1 | + | + | + | + |
| 5 × 10\hat{ }0 | + | + | − | − |
| Water | − | − | − | − |

The cloned GCC RNA was then subjected to in vitro mutagenesis to produce the Q version to be used for internal control and quantitation. The Q version of GCC can be amplified using the same primer sets as used for amplifying WT GCC, but differs from WT GCC by a substitution of 16 nucleotides in the region of the detection probe. The Q RNA therefore does not hybridize to the WT probe A, and the WT RNA does not hybridize to the Q probe. (Data not shown).

For a quantitative assay, a known amount of Q RNA is spiked into the sample and then subjected to extraction and amplification along with the sample RNA. After amplification, the products were independently probed with the WT probe A and the Q probe. The amount of WT RNA present is calculated from the ratio obtained of Q signal to WT signal. This signal ratio was tested in the assay using primer set BB to amplify known quantities of in vitro transcribed WT RNA. The results in Table 7 show that GCC RNA can be quantitated using the NASBA method.

TABLE 7

| Input WT in vitro RNA | Mean of Calculated WT RNA (n = 6) |
|---|---|
| 4.5 × 10\hat{ }6 | 3.6 × 10\hat{ }6 |
| 4.5 × 10\hat{ }5 | 4.9 × 10\hat{ }5 |
| 4.5 × 10\hat{ }4 | 4.5 × 10\hat{ }4 |
| 4.5 × 10\hat{ }3 | 5.7 × 10\hat{ }3 |
| Water | <lower limit |

The GCC quantitative NASBA assay has also been applied to the quantitation of GCC RNA in cells. HT-29 and T-84 cell line dilutions were used as template and were amplified using the BB primer set. (Data not shown).

The results shown in the present application demonstrate that the primers and probes of the present invention can specifically detect low levels of target molecule, even in the background of clinical samples. In addition, the primers can amplify the Q RNA, while the Q probe and WT probe A hybridize specifically to their cognate targets. Thus, the primers used in the present invention provide unexpectedly good results for the detection and quantitation of GCC RNA.

The publications and other materials used herein to illuminate the background of the invention, and provide additional details respecting the practice of the invention, are incorporated herein by reference as if each was individually incorporated herein be reference.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

What is claimed is:

1. An oligonucleotide selected from the group consisting of SEQ ID NO:3, SEQ ID NO:4, SEQ ID NO:7, SEQ ID NO:8, SEQ ID NO:9, SEQ ID NO: 10 and SEQ ID NO:11.

2. The oligonucleotide of claim 1, wherein the oligonucleotide is selected from the group consisting of SEQ ID NO:3, SEQ ID NO:4, SEQ ID NO:7, and SEQ ID NO:8.

3. An oligonucleotide of about 15-26 nucleotides, comprising at least 10 consecutive nucleotides of a sequence selected from the group consisting of SEQ ID NO:3, SEQ ID NO:4, SEQ ID NO:7, SEQ ID NO:8, SEQ ID NO:9 SEQ ID NO:10 and SEQ ID NO:11.

4. The oligonucleotide of claim 3, wherein the oligonucleotide is selected from the group consisting of SEQ ID NO:3, SEQ ID NO:4, SEQ ID NO:7, and SEQ ID NO:8.

5. A method for the detection or quantitation of GCC RNA in a sample, comprising:
a) obtaining a sample which may contain GCC RNA;
b) performing isothermal transcription based amplification on the sample with two oligonucleotide primers, a first primer which comprises at least 10 consecutive nucleotides of a sequence selected from the group consisting of SEQ ID NO:3 and SEQ ID NO:4, and a second primer which comprises at least 10 consecutive nucleotides of a sequence selected from the group consisting of SEQ ID NO:7 and SEQ ID NO:8; and
c) detecting or quantitating the resulting product of step b), whereby detection or quantitation of the amplification product indicates the presence or quantity of GCC RNA in the sample.

6. The method of claim 5, wherein detection of the amplification product uses a labelled wild-type probe comprising a sequence according to SEQ ID NO:9, whereby hybridization of the wild-type probe to the amplification product indicates the presence of GCC RNA in the sample.

7. The method of claim 6, further comprising adding a known amount of control RNA Q at step b), and detecting amplification product of Q by using a labeled probe comprising the sequence of SEQ ID NO:11, whereby the quantity of GCC RNA in the sample is calculated by comparing the signals of the probes for Q and the wild-type probe.

8. The method of claim 5, wherein the sample comprises cells and RNA is extracted from the cells in the sample prior to step b).

9. A primer pair for the detection or quantitation of GCC RNA in a sample, comprising one primer selected from the group consisting of SEQ ID NO:3 and SEQ ID NO:4, and one primer selected from the group consisting of SEQ ID NO:7 and SEQ ID NO:8.

10. The primer pair of claim 9, comprising SEQ ID NO:3 and SEQ ID NO:7.

11. A kit for the detection or quantitation of GCC RNA in a sample, comprising the primer pair of claim 9 and at least one probe selected from the group consisting of SEQ ID NO:9, SEQ ID NO:10, SEQ ID NO:11, and SEQ ID NO:12.

12. The oligonucleotide of claim 4, wherein the oligonucleotide is selected from the group consisting of SEQ ID NO:3 and SEQ ID NO:4, and the oligonucleotide further comprises a RNA polymerase promoter sequence operably attached to the 5′ end thereof.

13. The oligonucleotide of claim 12, wherein the RNA polymerase promoter sequence is a T7 RNA polymerase promoter as set forth in SEQ ID NO:1.

14. The method of claim 5, wherein the first primer further comprises a RNA polymerase promoter sequence operably attached to the 5′ end thereof.

15. The method of claim 14, wherein the RNA polymerase promoter sequence is a T7 RNA polymerase promoter as set forth in SEQ ID NO:1.

16. The method of claim 5, wherein the amplification product is detected using a capture probe comprising a sequence of SEQ ID NO:10.

17. A pair of oligonucleotides for the detection or quantitation of GCC RNA, a first oligonucleotide of said pair being about 15-26 nucleotides in length and comprising at least 10 consecutive nucleotides of a sequence selected from the group consisting of SEQ ID NO:3 and SEQ ID NO:4, and a second oligonucleotide of said pair being about 15-26 nucleotides in length and comprising at least 10 consecutive nucleotides of a sequence selected from the group consisting of SEQ ID NO:7 and SEQ ID NO:8.

18. The pair of oligonucleotides of claim 17, wherein the first oligonucleotide further comprises a RNA polymerase promoter sequence operably attached to the 5′ end thereof.

19. The pair of oligonucleotides of claim 18, wherein the RNA polymerase promoter sequence is a T7 RNA polymerase promoter sequence as set forth in SEQ ID NO:1.

20. The method of claim 5, wherein the isothermal transcription based amplification is nucleic acid sequence based amplification (NASBA).

21. A kit for the detection or quantitation of GCC RNA in a sample, comprising a pair of oligonucleotides of claim 17.

22. The kit of claim 21, further comprising at least one probe comprising a sequence selected from the group consisting of SEQ ID NO:9, SEQ ID NO:10, SEQ ID NO:11 and SEQ ID NO:12.

23. A kit for the detection or quantitation of GCC RNA in a sample, comprising a pair of oligonucleotides of claim 18.

24. The kit of claim 23, further comprising at least one probe comprising a sequence selected from the group consisting of SEQ ID NO:9, SEQ ID NO:10, SEQ ID NO:11 and SEQ ID NO:12.
